(19) United States
(12) Patent Application Publication
(10) Pub. No.: US 2026/0232493 A1
(43) Pub. Date: Aug. 13, 2026

(54) ANTI-MICROBIAL WOUND DRESSING AND METHODS OF USE THEREOF

(71) Applicant: Los Angeles Biosciences, Inc., Playa Del Rey, CA (US)

(72) Inventors: Derya Kelter, Los Angeles, CA (US); Lance Go, Fort Collins, CO (US)

(73) Assignee: Los Angeles Biosciences, Inc., Playa Del Rey, CA (US)

(21) Appl. No.: 19/533,650

(22) Filed: Feb. 9, 2026

Related U.S. Application Data
(60) Provisional application No. 63/758,031, filed on Feb. 13, 2025.

(51) Int. Cl. A61F 13/01; A61F 13/00

(52) U.S. Cl. CPC A61F 13/01029; A61F 13/00063; A61F 13/01017; A61F 13/01042; A61F 13/00987

(57) ABSTRACT

A device having a bioabsorbable component and a backing component. The bioabsorbable component is configured to contact the wound and includes an open cell matrix having a plurality of about 150 µm to about 500 µm pores. The backing component is joined to the bioabsorbable component directly or by an adhesive. One or more of the bioabsorbable component, the backing component, and the adhesive may include a plurality of one or more of nanoparticles and salts that may be released through the open cell matrix and into the wound to promote healing. The backing component is removable from the bioabsorbable component after the plurality of one or more of nanoparticles and salts are deposited into the wound and the bioabsorbable component may be retained over the wound.

### CROSS-REFERENCE TO RELATED APPLICATIONS

This application claims benefit to U.S. Prov. Pat. App. No. 63/758,031 filed on February 13, 2025. The entirety of this application is incorporated herein by reference.

### TECHNICAL FIELD

The present disclosure relates generally to methods, systems, and apparatuses related to covering a wound. More particularly, the present disclosure relates to a device capable of being secured over a wound and promoting healing using one or more of nanoparticles and salts (e.g., of a metal cation and/or alloys thereof). The disclosed techniques may be applied to, for example, treating wounds in dermal tissue (e.g., burns).

### BACKGROUND

Wound dressings may be a beneficial tool for effectively treating wounds in dermal tissue, including but not limited to burns. For example, wound dressings may be effective for protecting the wound from re-injury as well as keeping the wound clean to prevent infection during the initial stages of healing.

Conventional wound dressings include a covering material for the wound site formed from a natural material. The covering material may need to be changed in regular increments. However, this process may partially disrupt the wound site and limit the degree of healing (e.g., leading to scarring). Furthermore, wound care solutions and topical formulations may need to be separately applied to the wound in order to prevent infection. Ideally, a wound dressing could be suited to promote the body’s natural recovery mechanisms while mitigating wound site disruption and/or infection.

As such, it is important to cover and treat wounds in an effective manner that allows for optimal recovery of the tissue. Accordingly, it would be advantageous to have a wound covering device configured to facilitate tissue ingrowth and angiogenesis at the wound site.

### SUMMARY

A device for covering a wound in a dermal tissue is provided. The device may include a bioabsorbable component having a first surface configured to contact the wound and a second surface opposing the first surface, wherein the bioabsorbable component may include an open cell matrix having a plurality of pores. In an example, the plurality of pores may have diameters of about 150 µm to about 500 µm and the bioabsorbable component may include a thickness of about 0.5 mm to about 2.0 mm. The device may further include a backing component joined to the second surface and impregnated with a plurality of one or more of nanoparticles and salts (e.g., of metal cation and/or alloys thereof). The one or more of nanoparticles and salts may be configured to be released through the open cell matrix and into the wound. The nanoparticles may have a diameter of about 1 nm to about 100 nm. The salts may have a diameter of about 0.1 µm to about 100 µm. In an example, the device may further include an adhesive between the bioabsorbable component and the backing component. In another example, the bioabsorbable component and the backing component may be directly bonded (e.g., thermally bonded or covalently bonded) to one another. The backing component may be removable from the bioabsorbable component after the plurality of one or more of nanoparticles and salts are deposited into the wound.

According to some examples, the bioabsorbable component may be formed from a synthetic material. According to additional examples, the bioabsorbable component may be formed from a polyester polyurethane or a derivative thereof. According to further examples, the polyester polyurethane or the derivative thereof may be a non-aromatic isocyanate polyester polyurethane or a derivative thereof.

According to some examples, the bioabsorbable component may be formed as a bilayer of open cell matrix.

According to some examples, the backing layer may include a laminate material. According to additional examples, the laminate material may include a fenestrated laminate material.

A device for covering a wound in a tissue is also provided. The device may include a bioabsorbable component having a first surface configured to contact the wound and a second surface opposing the first surface, and the bioabsorbable component may include an open cell matrix having a plurality of pores. In an example, the plurality of pores may have diameters of about 150 µm to about 500 µm. The device may further include a backing component joined to the second surface and impregnated with a plurality of one or more of nanoparticles and salts. The nanoparticles may have a diameter of about 1 nm to about 100 nm. The salts may have a diameter of about 0.1 µm to about 100 µm. The one or more of nanoparticles and salts may be configured to be released through the open cell matrix and deposited into the wound, thereby promoting healing of the wound. In an example, the device may further include an adhesive adhering the backing component to the bioabsorbable component. In another example, the bioabsorbable component and the backing component may be directly bonded (e.g., thermally bonded or covalently bonded) to one another. The backing component may be removable from the bioabsorbable component after the plurality of one or more of nanoparticles and salts are deposited into the wound.

According to some examples, the bioabsorbable component may be formed from a synthetic material. According to additional examples, the bioabsorbable component may be formed from a polyester polyurethane or a derivative thereof. According to further examples, the polyester polyurethane or the derivative thereof may include a non-aromatic isocyanate polyester polyurethane or a derivative thereof.

According to some examples, the bioabsorbable component may have a thickness of about 0.5 mm to about 1.5 mm.

According to some examples, the bioabsorbable component may have a thickness of about 1.5 mm to about 5 mm.

According to some examples, the bioabsorbable component may be formed as a bilayer of open cell matrix.

According to some examples, the backing layer may include a laminate (e.g. fenestrated) material.

According to some examples, the plurality of one or more of nanoparticles and salts may include nanoparticles or salts of a metal cation and/or alloys thereof.

According to some examples, the bioabsorbable component may include a plurality of struts disposed within the open cell matrix. The plurality of struts may be configured to increase the tensile strength of the bioabsorbable component.

According to some examples, the bioabsorbable component may include an additional plurality of one or more of nanoparticles and salts that may be impregnated in the open cell matrix. The additional plurality of nanoparticles may have a diameter of about 1 nm to about 100 nm. The additional plurality of salts may have a diameter of about 0.1 µm to about 100 µm. The additional plurality of one or more of nanoparticles and salts may be configured to be released through the open cell matrix and deposited into the wound, thereby promoting healing of the wound.

A method of treating a wound in a dermal tissue is also provided. The method may include placing a device in contact with the wound. The device may include: a bioabsorbable component having a first surface contacting the wound and a second surface opposing the first surface, wherein the bioabsorbable component may include an open cell matrix including a plurality of pores. The plurality of pores may have diameters of about 150 µm to about 500 µm and the bioabsorbable component may have a thickness of about 0.5 mm to about 2.0 mm. A backing component may be joined to the second surface and impregnated with a plurality of one or more of nanoparticles and salts (e.g., of metal cation and/or alloys thereof). The nanoparticles may have a diameter of about 1 nm to about 100 nm. The salts may have a diameter of about 0.1 µm to about 100 µm. In an example, an adhesive may be between the bioabsorbable component and the backing component. In another example, the bioabsorbable component and the backing component may be directly bonded (e.g., thermally bonded or covalently bonded) to one another. The method may further include retaining the device over the wound for a first time period, wherein the one or more of nanoparticles and salts may be configured to be released through the open cell matrix and into the wound during the first time period, thereby promoting healing of the wound. The method may further include removing the backing component from the bioabsorbable component after the first period and retaining the bioabsorbable component following the first time period, thereby promoting angiogenesis in the dermal tissue

### BRIEF DESCRIPTION OF THE DRAWINGS

The accompanying drawings, which are incorporated in and form a part of the specification, illustrate the examples of the invention and together with the written description serve to explain the principles, characteristics, and features of the invention. Various aspects of at least one example are discussed below with reference to the accompanying drawings, which are not intended to be drawn to scale. In the drawings:

FIG. 1A depicts a diagrammatic view of a device for covering a wound, according to an example of the present disclosure;

FIG. 1B depicts a diagrammatic view of a device for covering a wound, according to an example of the present disclosure;

FIG. 1C depicts a diagrammatic view of a device for covering a wound, according to an example of the present disclosure; and

FIG. 2 depicts a flow diagram of an illustrative method of treating a wound in a dermal tissue using a device as disclosed herein, according to an example of the present disclosure.

### DETAILED DESCRIPTION

As discussed herein, it would be advantageous to have a wound covering device configured to cover and treat wounds in an effective manner that allows for optimal recovery of the tissue. Ideally, the device would facilitate tissue ingrowth and angiogenesis at the wound site. In some examples, the device may be configured to activate the body’s physiological healing mechanisms in a directed manner to build vascular structures throughout the tissue as it heals.

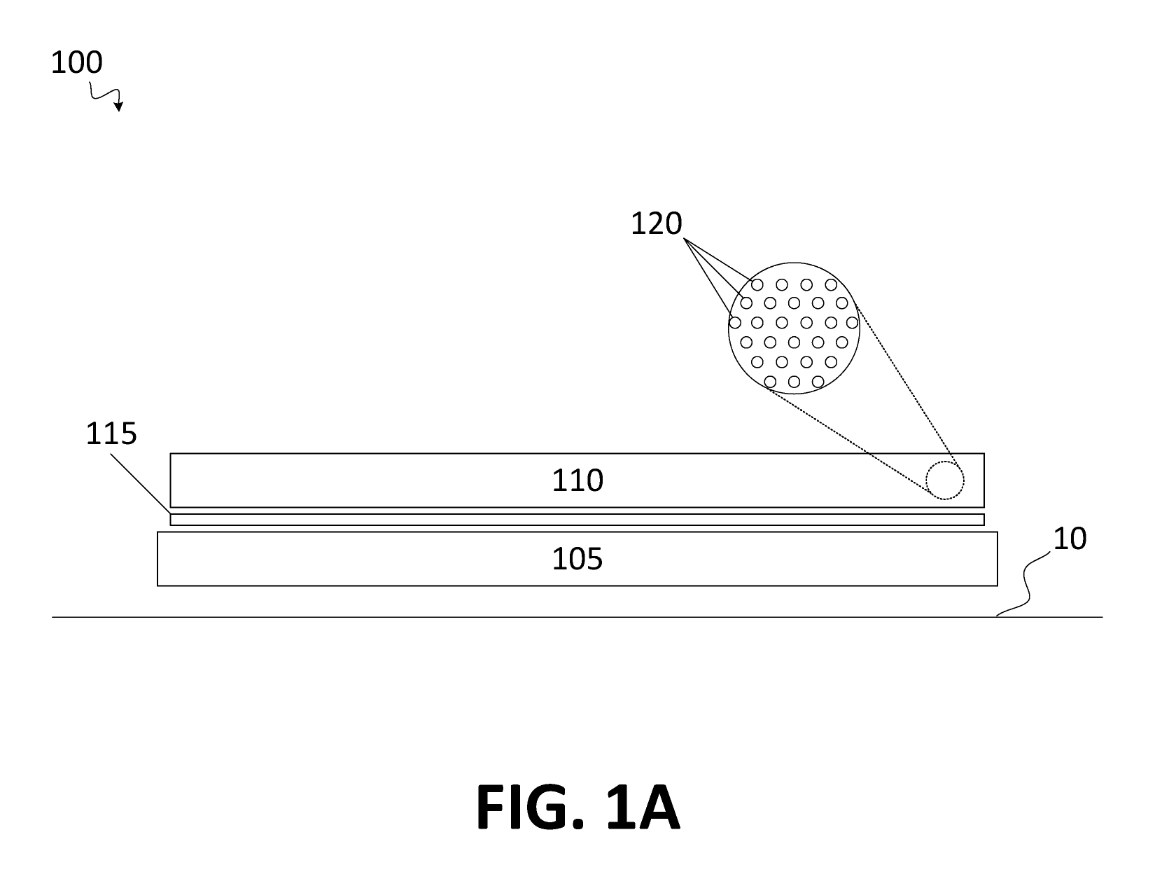

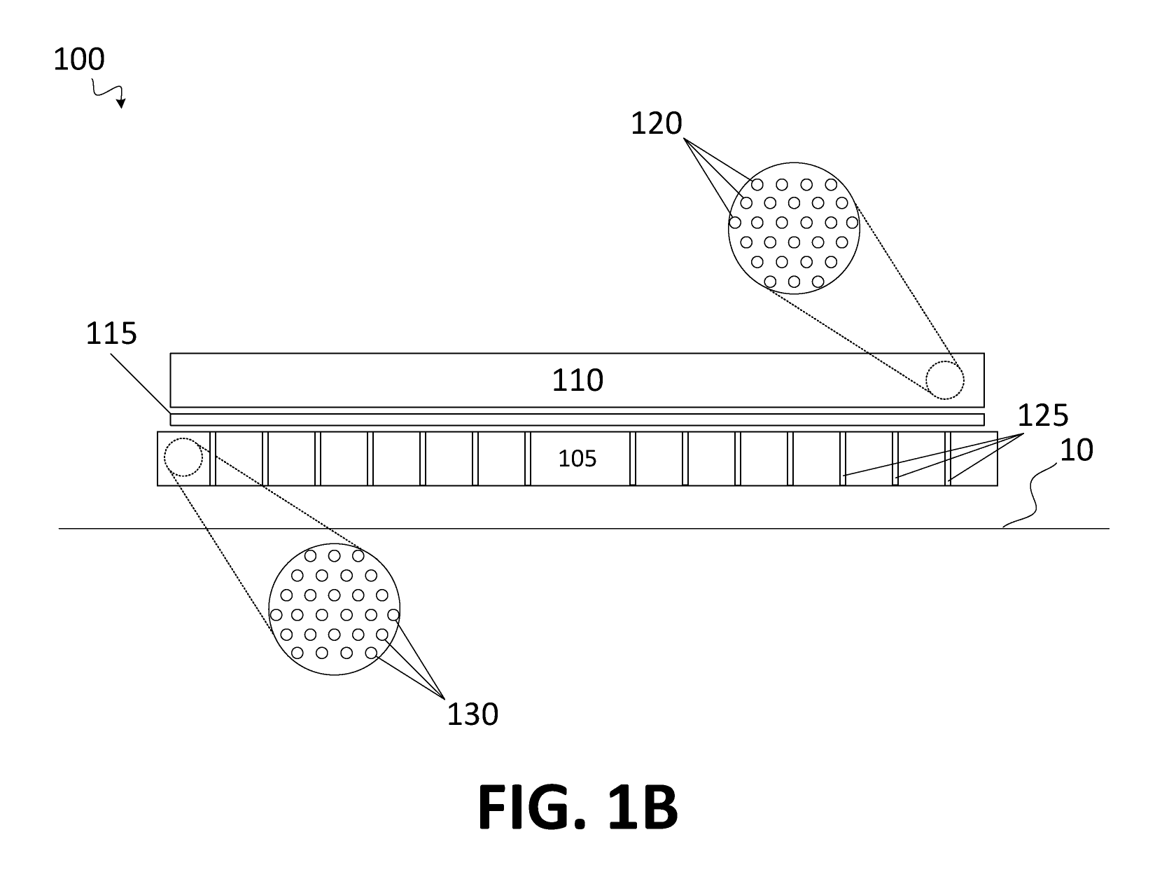

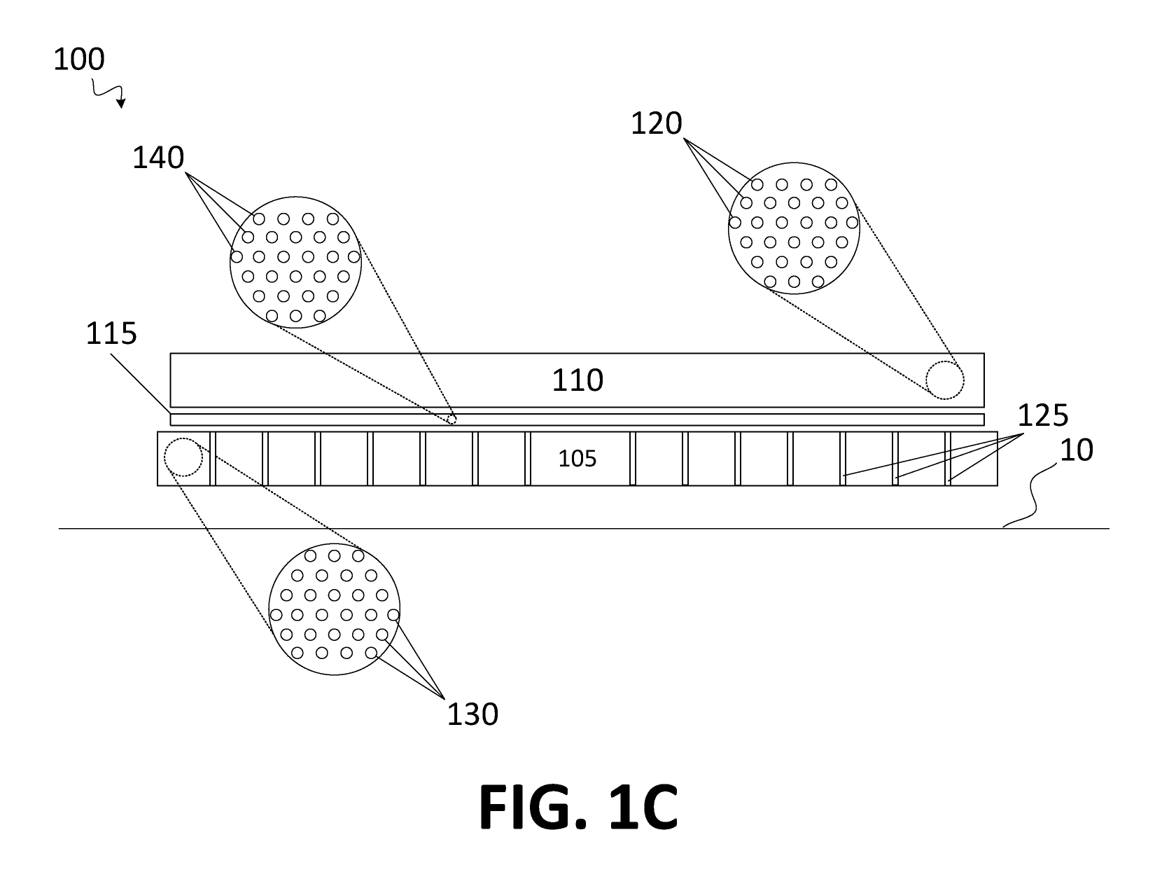

FIG. 1A depicts a diagrammatic view of a device for covering a wound according to an example of the present disclosure. FIG. 1B depicts a diagrammatic view of a device for covering a wound according to another example of the present disclosure. FIG. 1C depicts a diagrammatic view of a device for covering a wound according to another example of the present disclosure. Similar features within FIGS. 1A-1C are identified with common reference numbers.

As shown in FIG. 1A, the device 100 may include a bioabsorbable component 105, a backing component 110, optionally an adhesive 115, and a therapeutic component 120. In some examples, therapeutic component 120 may be disposed within the backing component 110. In some examples, as shown in FIG. 1B, the device 100 may further include a plurality of struts 125 and/or a second therapeutic component 130 disposed within the bioabsorbable component 105. In some examples, as shown in FIG. 1C, the device 100 may further include a third therapeutic component 130 disposed within the adhesive 115.

The bioabsorbable component 105 may define a first layer of the device 100 configured to contact the wound 10. The bioabsorbable component 105 may be formed as a bilayer of open cell matrix, i.e., two stacked layers of material forming the open cell matrix. However, in additional examples, a single layer of material or two or more layers of material may be utilized.

The bioabsorbable component 105 may include a first surface configured to contact the wound site and a second surface opposing the first surface. The second surface may contact additional components of the device 100, e.g., the backing component 110 and/or the adhesive 115. Accordingly, the bioabsorbable component 105 may be arranged as the bottom-most layer of the device 100 in order to contact the wound 10 directly when placed thereover.

In some examples, the open cell matrix may be a foam material. In some examples, the open cell matrix may be formed from a synthetic foam material. The material may be selected to provide a sufficient structure to the wound to promote healing, tissue in-growth, and/or angiogenesis. Furthermore, the material may be selected to be minimally reactive in the tissue and/or to be sufficiently biodegradable (e.g., hydrolysable) in the body such that it may be left in place on the wound 10 and naturally degraded over time as healing progresses. For example, the open cell matrix may be formed from a polyurethane foam. In a particular example, the polyurethane foam may be a polyester polyurethane or a derivative thereof, e.g., a non-aromatic isocyanate polyester polyurethane or a derivative thereof.

In additional examples, the material of the open cell matrix may include one or more of a polycaprolactone, a polydioxanone, a polyglycolic acid, a polyhydroxybutyrate, a polyhydroxyvalerate, a polylactic acid bases polymer, a synthetic non-aromatic polymer, or another synthetic material with sufficient characteristics as described herein as would be known to a person having an ordinary level of skill in the art. The open cell matrix may include biodegradable polyesters (e.g., polycaprolactone, polydioxanone, polyglycolic acid, polyhydroxyburyrate, polyhydroxyvalerate, polylactic acid, etc.) formed on polyurethane or with polyurethane linkages. The open cell matrix may include a copolymer of polylactic acid and polycaprolactone to form a flexible foam. In other examples, the open cell matrix may be a photocurable resin (e.g. polyurethane acrylates). The open cell matrix may be a copolymer of any of these materials, such as poly(lactic acid-co-glycolic acid), poly(glycolic acid-co-caprolactone), or poly(hydroxybutyrate-co-hydroxyvalerate).

The open cell matrix of the bioabsorbable component 105 may include a microstructure configured to promote angiogenesis in the tissue of the wound 10. In some examples, the bioabsorbable component 105 may include a plurality of pores formed in the open cell matrix. In some examples, the plurality of pores may have diameters of about 150 µm to about 500 µm. It should be understood that the pore size may be selected based on known biologic and physiologic factors in order to particular promote optimal ingrowth of tissue and/or angiogenesis. A pore size of about 150 µm to about 500 µm may be beneficial in particular contexts, e.g., burns in dermal tissue as well as other types of wounds. This particular pore size may be adequate for enabling angiogenesis to occur within the open cell matrix, thereby promoting healing of the tissue. In some examples, additional ranges may be selected based on the particular type of tissue and/or type of wound. For example, the pores may have a diameter of about 150 µm, about 160 µm, about 170 µm, about 180 µm, about 190 µm, about 200 µm, about 210 µm, about 220 µm, about 230 µm, about 240 µm, about 250 µm, about 260 µm, about 270 µm, about 280 µm, about 290 µm, about 300 µm, or individual values or ranges therebetween. In some examples, the pores may have varying sizes, e.g., a first set of pores may have a first pore size from amongst the disclosed sizes and ranges while a second set of pores may have a second pore size from amongst the disclosed sizes and ranges. The pores may be tightly packed within the material of the bioabsorbable component 105 at a density that approximates the microvasculature of human tissue. In some examples, the density of the pores may be selected based on the type of tissue of the wound in order to direct healing and angiogenesis in a manner that mimics the tissue.

In some examples, the open cell matrix of the bioabsorbable component 105 may have a thickness of about 0.5 mm to about 2.0 mm. However, the thickness of the bioabsorbable component 105 may be selected based on the desired tensile strength of the device 100. For example, the tensile strength may be selected based on the size of the wound 10, the location of the wound 10, the type of tissue at the site of the wound 10, and/or the depth of the wound 10. Based on these factors, a tensile strength may be selected to provide adequate stability, promote tissue ingrowth, and enable angiogenesis within the tissue. In some examples, the bioabsorbable component 105 may have a thickness of about 0.5 mm, about 1 mm, about 1.5 mm, about 2 mm, about 2.5 mm, about 3 mm, about 3.5 mm, about 4 mm, about 4.5 mm, about 5 mm, greater than about 5 mm, or individual values or ranges therebetween.

In some examples, the bioabsorbable component 105 may further include a mesh layer. For example, a mesh layer may be included above the open cell matrix, below the open cell matrix, and/or between layers of the open cell matrix. The mesh layer may increase the tensile strength of the bioabsorbable component 105 to provide adequate stability, promote tissue ingrowth, and/or enable angiogenesis within the tissue for a given application.

In some examples, the bioabsorbable component 105 may include a plurality of struts 125 within the open cell matrix in order to increase the tensile strength of the bioabsorbable component 105. For example, as shown in FIG. 1B, the struts 125 may be dispersed throughout the bioabsorbable component 105 at regular increments. The struts 125 may promote healing in the tissue in a directed fashion by modifying the physiological mechanisms that occur at a wound site. For example, the struts 125 may break up collagen accumulation and/or prevent myofibroblast bands from forming in the wound 10, thereby preventing contraction of the wound and increasing vascularity as angiogenesis occurs in the tissue. Furthermore, the struts 125 may increase the structural strength of the tissue by directing healing in the described manner. The presence of the struts 125 may also increase the tensile strength of the bioabsorbable component 105 to provide adequate stability, promote tissue ingrowth, and/or enable angiogenesis within the tissue for a given application.

The backing component 110 may be joined to the second surface of the bioabsorbable component 105. For example, as shown in FIGS. 1A-1C, the adhesive 115 may be disposed between the backing component 110 and the bioabsorbable component 105 to secure the backing component 110 to the bioabsorbable component 105. In some examples, the adhesive 115 may have an adequate strength such that the backing component 110 may be later removed from the bioabsorbable component 105 as further described herein. In another example, the bioabsorbable component 105 and the backing component 110 may be directly bonded (e.g., thermally bonded or covalently bonded) to one another.

The backing component 110 may be formed from a laminate material. For example, the backing component 110 may be formed from a laminate material. The laminate material may be fenestrated. The laminate material may be melted. However, the backing component 110 may be formed from additional materials with sufficient characteristics as described herein as would be known to a person having an ordinary level of skill in the art.

The therapeutic component 120 may be disposed within the backing component 110. In some examples, the therapeutic component 120 may be a solution of one or more of nanoparticles and salts of a metal cation and/or alloys thereof. For example, the therapeutic component 120 may provide one or more of antimicrobial, healing, and angiogenic properties and may include one or more of silver, zinc, calcium, magnesium, iron, zirconium, copper, and mixtures or alloys thereof (e.g., zinc-ferrite, magnesium-zinc, magnesium-silver, silver zirconium phosphate, etc.). In an example, the therapeutic component 120 may include one or more of silver nanoparticles, silver sulfate, silver chloride, silver sulfadiazine, silver sodium hydrogen zirconium phosphate, etc. Silver ions may be capable of functioning as a non- specific, broad anti-microbial compound in order to prevent infection from a wide array of bacteria and/or fungi. Furthermore, silver ions may not provoke anti-microbial resistance over time. Still further, silver ions may not interfere with the function of human fibroblasts and/or the wound healing process. However, in additional examples, the therapeutic component 120 may include a wide variety of compounds known to deter infection as would be apparent to a person having an ordinary level of skill in the art, e.g., antibiotic compounds, anti-septic agents, other anti-microbial ions (e.g., copper), small molecule inhibitors, and the like. In some examples, the one or more of nanoparticles and salts may have a diameter of about 1 nm to about 100 nm.

The therapeutic component 120 may be impregnated in the backing component 110 and/or coated on the backing component 110 such that, after the device 100 is placed over the wound 10, the therapeutic component 120 may be eluted into the open cell matrix of the bioabsorbable component 105 and/or into the wound 10, thereby deterring infection and promoting healing of the wound 10. In some examples, the backing component 110 may be removed from the bioabsorbable component 105 after the therapeutic component is eluted through the open cell matrix and deposited in the tissue. Thereafter, the bioabsorbable component 105 may be retained over the wound 10 as healing progresses.

As shown in FIG. 1B, in some examples, a second therapeutic component 130 may be disposed in the bioabsorbable component 105. The second therapeutic component 130 may reinforce the function of the therapeutic component 120 and provide greater resistance to microbial infection. The second therapeutic component 130 may include any of the compounds described with respect to the therapeutic component 120. In some examples, the second therapeutic component 130 may be the same as the therapeutic component 120, e.g., the one or more of nanoparticles and salts. In additional examples, the second therapeutic component 130 may be a different compound than the therapeutic component 120.

As shown in FIG. 1C, in some examples, a third therapeutic component 140 may also be disposed in the adhesive 115. The third therapeutic component 140 may reinforce the function of the therapeutic component 120 and/or the second therapeutic component 130 and may provide greater resistance to microbial infection. The third therapeutic component 140 may include any of the compounds described with respect to the therapeutic component 120. In some examples, the third therapeutic component 140 may be the same as the therapeutic component 120, e.g., the one or more of nanoparticles and salts. In additional examples, the third therapeutic component 140 may be a different compound than the therapeutic component 120.

While FIG. 1C shows an example of the device 100 with the therapeutic component 120, the second therapeutic component 130, and the third therapeutic component 140 present, any combination of the therapeutic component 120, the second therapeutic component 130, and the third therapeutic component 140 is contemplated. Further, while the struts 125 are shown in FIG. 1C, it is contemplated that any combination of any combination of the therapeutic component 120, the second therapeutic component 130, and the third therapeutic component 140 may be used with or without the struts 125.

In some examples, one or more of the therapeutic component 120, the second therapeutic component 130, and the third therapeutic component 140 may be at a concentration of about 4%. However, additional concentrations are contemplated as would be apparent to a person having an ordinary level of skill in the art. In some instances, one or more of the therapeutic component 120, the second therapeutic component 130, and the third therapeutic component 140 may be at concentration of about 0.1 % to greater than about 7%, or individual values or ranges therebetween.

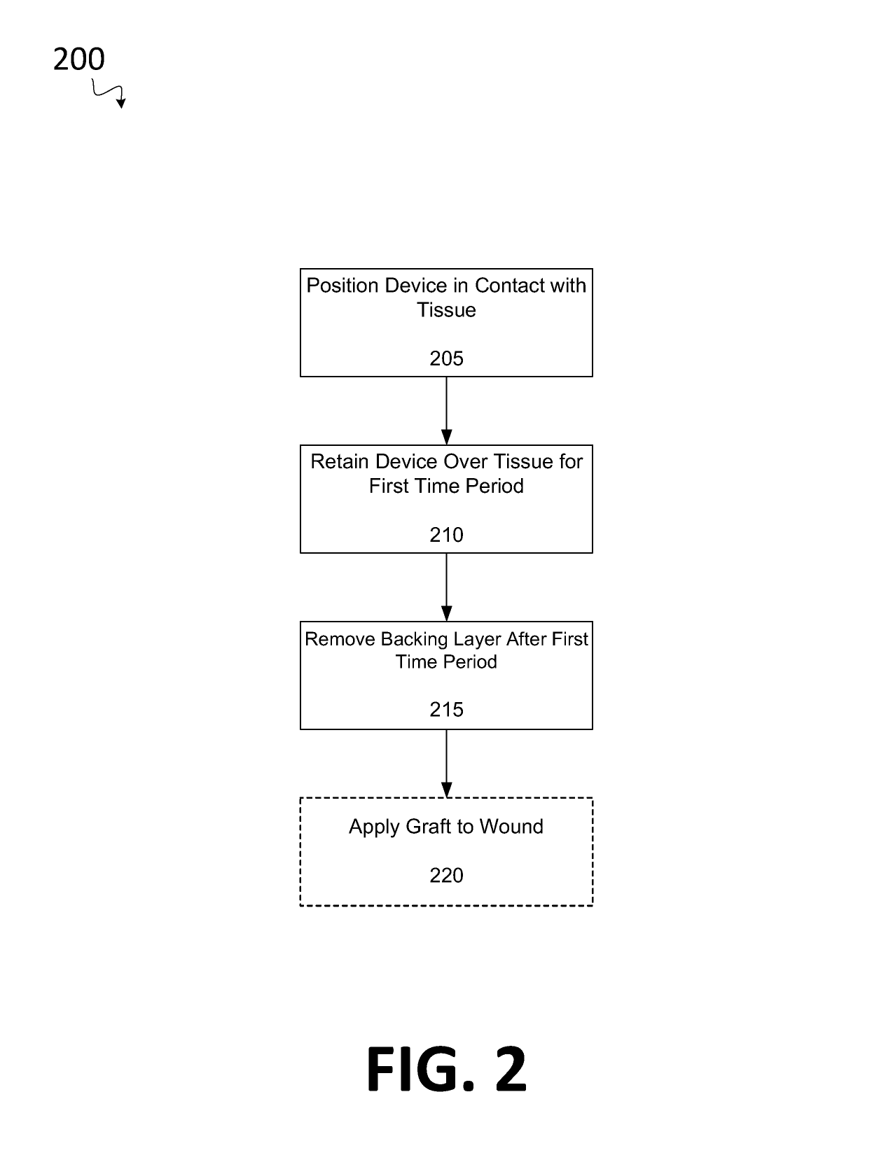

Referring now to FIG. 2, an illustrative method of treating a wound in a dermal tissue using a wound covering device as disclosed herein is depicted in accordance with an example. For example, the wound covering device may be a device 100 as described herein. As shown, the method 200 may include positioning 205 the device in contact with a tissue, retaining 210 the device over the tissue for a first time period, and removing 215 a backing layer from the device after the first time period. In some examples, the method 200 may further include applying 220 a graft to the wound after removing the backing layer.

As shown in FIGS. 1A-1B, the device 100 may be positioned 205 over the tissue, e.g., a dermal tissue, to treat a wound 10. The device 100 may be positioned 205 by aligning the bioabsorbable component 105 with the tissue such that the wound 10 is within the span of the bioabsorbable component 105 and thereby covered. In some examples, the tissue 10 may be prepared for covering in a variety of manners. For example, a dermal tissue may be prepared by cleaning the area with an anti-microbial compound as would be known to a person having an ordinary level of skill in the art.

The device 100 may be secured over the wound 10 in a variety of manners. In some examples, the lower surface of the device 100 (i.e., the first surface of the bioabsorbable component 105) may include adhesive portions at the opposing ends in order to secure over the tissue. In some examples, the device 100 may be secured by wrapping over the tissue with a dressing material, e.g., gauze or another dressing as would be known to a person having an ordinary level of skill in the art.

The device 100 may be retained 210 over the tissue for a first time period. In some examples, during the first time period, the therapeutic component 120 and/or the additional therapeutic component 130 may be eluted from the respective portions of the device 100 into the wound. Accordingly, the device 100 may prevent infection in the wound 10 during healing. Furthermore, during the first time period, the bioabsorbable component 105 may provide structure (e.g., the open cell matrix of particular thickness and particular pore size) that promotes an engineered response by the bed of the wound 10 including tissue ingrowth within the bioabsorbable component 105 and angiogenesis to form vascular structure around and through the bioabsorbable component 105. Accordingly, the wound 10 may be directed towards forming highly vascular granulation-type tissue into and within the foam structure. The structure of the open cell matrix may also deter formation of confluent bands of myofibroblasts that might otherwise cause wound contracture due to the physical disruption in the tissue by the bioabsorbable component 105.

After the first time period, whereby the therapeutic component 120 has eluted into the wound 10, the backing component 110 may be removed from the wound 10, e.g., by peeling away from the bioabsorbable component 105. Thereafter, the bioabsorbable component 105 may be retained on the wound 10. As described, tissue ingrowth may occur around and through the open cell matrix of the bioabsorbable component 105 and may continue to progress after the removal of the backing component 110.

In some examples, after the wound 10 has healed sufficiently to grow tissue and form vascular structures through the bioabsorbable component 105, a graft may be applied 220 to the wound 10 to further recover the tissue. For example, in the context of a dermal wound (e.g., a burn), a skin graft may be applied 220 to the wound to replace the lost layers of dermal tissue. The healing process promoted by the device 100 as described herein may ready the dermal tissue to receive the skin graft and to rapidly incorporate the skin graft into the dermal tissue. Furthermore, the healing process promoted by the device 100 may reduce the presence and/or the severity of scarring in the areas surrounding the graft.

The devices, systems, and methods as described herein are not intended to be limited in terms of the particular examples described, which are intended only as illustrations of various features. Many modifications and variations to the devices, systems, and methods can be made without departing from their spirit and scope, as will be apparent to those skilled in the art.

The devices and methods disclosed herein may be applied to a variety of burns, e.g., partial and/or full thickness burns. Furthermore, while the present disclosure makes specific reference to burn wounds, it should be understood that the disclosed devices and methods may be applied to a variety of types of wounds in a variety of tissues. For example, the devices and methods disclosed herein may be applied to traumatic wounds (e.g., abrasions and/or lacerations), post-surgical wounds (e.g., tissues having undergone excision and/or debridement for necrotizing infections), and other types of wounds.

Furthermore, while external use is generally described herein, the devices and methods disclosed herein may also be applied to the context of internal wounds and may be configured for implantation within the body with reasonable modifications (e.g., removal of the non-biodegradable backing layer). For example, the devices and methods disclosed herein may be used for the purpose of reducing scar contraction along an implant surface or tissue plane such as around breast implants, around implanted pacemakers, around joints, and/or over tendons. The disclosed devices and methods may also be used for reinforcing tissue planes subject to stretching such as breast tissue after a breast lift or tissue around a hernia repair.

In the context of internal implantation, the superstructure of the bioabsorbable component 105 may be modified. For example, while a bilayer design is described herein, a single layer may be utilized in internal applications. In another example, while a thickness of at least about 0.5 mm for the open cell matrix is described herein, a thinner foam layer may be utilized for internal applications. In some examples, the open cell matrix may have a thickness of about 0.5 mm, about 0.4 mm, about 0.3, mm, about 0.2 mm, about 0.1 mm, less than about 0.1 mm, or individual values or ranges therebetween. Furthermore, a mesh layer may be utilized as part of the bioabsorbable component 105 as described herein particularly in applications involving internal implantation in order to increase the tensile strength as may be required in this context.

Additionally, in the context of internal implantation, the device may be utilized without a backing component 110 because the backing component 110 may hinder incorporation and healing in an internal environment. Omission of the backing component 110 may lead to more rapid incorporation of the bioabsorbable component 105 into the tissue structure through tissue ingrowth and angiogenesis. In examples omitting the backing component 110, the second therapeutic component 130 may be incorporated into the bioabsorbable component 105 (e.g., as shown in FIG. 1B) and/or a separate non-removable layer may be incorporated over the bioabsorbable component to hold the second therapeutic component 130. In examples omitting the backing component 110, the concentration of the second therapeutic component 130 in the bioabsorbable component and/or the separate layer may be higher. Such examples may be particularly useful for application around breast implants and pacemakers to decrease the risk of scar capsule contracture and/or provide prolonged antimicrobial compound elution in the peri-implant space to decrease infection risk.

Furthermore, in the context of internal implantation, an organic anti-microbial compound may be utilized for the therapeutic component 120 and/or second therapeutic component 130. For example, while nanoparticles are disclosed as an exemplary anti-microbial compound, an organic compound may be advantageous when the device 100 is implanted internally to prevent immune response.

In the above detailed description, reference is made to the accompanying drawings, which form a part hereof. In the drawings, similar symbols typically identify similar components, unless context dictates otherwise. The illustrative examples described in the present disclosure are not meant to be limiting. Other examples may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular examples described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular examples described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular examples only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed examples.

This disclosure is not limited to the particular systems, devices and methods described, as these may vary. The terminology used in the description is for the purpose of describing the particular versions or examples only, and is not intended to limit the scope. Such aspects of the disclosure be embodied in many different forms; rather, these examples are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample examples, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios of a composition are based upon the total weight of the composition and all measurements made are at about 25 ºC, unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the example of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

Unless defined otherwise, all technical and scientific terms used herein have the same meanings as commonly understood by one of ordinary skill in the art. Nothing in this disclosure is to be construed as an admission that the examples described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

What is claimed is:

1. A device for covering a wound in a dermal tissue, the device comprising:
a bioabsorbable component having a first surface configured to contact the wound and a second surface opposing the first surface, wherein the bioabsorbable component comprises an open cell matrix including a plurality of pores having diameters of about 150 µm to about 500 µm; and
a backing component joined to the second surface and comprising a plurality of one or more of nanoparticles and salts configured to be released through the open cell matrix and into the wound, the nanoparticles having a diameter of about 1 nm to about 100 nm and the salts having a diameter of about 0.1 µm to about 100 µm, wherein the backing component is removable from the bioabsorbable component after the plurality of one or more of nanoparticles and salts are deposited into the wound.

2. The device of claim 1, wherein the bioabsorbable component further comprises the one or more of nanoparticles and salts.

3. The device of claim 1, further comprising:
an adhesive between the bioabsorbable component and the backing component.

4. The device of claim 3, wherein the adhesive comprises the one or more of nanoparticles and salts.

5. The device of claim 1, wherein the bioabsorbable component is formed from a polyester polyurethane or a derivative thereof.

6. The device of claim 1, wherein the bioabsorbable component is formed as a bilayer of the open cell matrix.

7. The device of claim 1, wherein the backing component comprises a laminate material.

8. The device of claim 1, wherein the bioabsorbable component comprises a thickness of about 0.5 mm to about 5 mm.

9. The device of claim 1, wherein the plurality of one or more of nanoparticles and salts comprise one or more of a metal cation and/or alloys thereof.

10. The device of claim 1, wherein the bioabsorbable component further comprises a plurality of struts disposed within the open cell matrix.

11. A method of treating a wound in a dermal tissue, the method comprising:
placing a device in contact with the wound, the device comprising:
a bioabsorbable component having a first surface contacting the wound and a second surface opposing the first surface, wherein the bioabsorbable component comprises an open cell matrix including a plurality of pores having diameters of about 150 µm to about 500 µm, and
a backing component joined to the second surface and impregnated with a plurality of one or more of nanoparticles and salts, the nanoparticles having a diameter of about 1 nm to about 100 nm and the salts having a diameter of about 0.1 µm to about 100 µm;
retaining the device over the wound for a first time period, wherein the one or more of nanoparticles and salts are configured to be released through the open cell matrix and into the wound during the first time period, thereby promoting healing of the wound; and
removing the backing component from the bioabsorbable component after the first period and retaining the bioabsorbable component following the first time period, thereby promoting angiogenesis in the dermal tissue.

12. The method of claim 11, wherein the bioabsorbable component further comprises the one or more of nanoparticles and salts.

13. The method of claim 11, wherein the device further comprises:
an adhesive between the bioabsorbable component and the backing component.

14. The method of claim 13, wherein the adhesive comprises the one or more of nanoparticles and salts.

15. The method of claim 11, wherein the bioabsorbable component is formed from a polyester polyurethane or a derivative thereof.

16. The method of claim 11, wherein the bioabsorbable component is formed as a bilayer of the open cell matrix.

17. The method of claim 11, wherein the backing component comprises a laminate material.

18. The method of claim 11, wherein the bioabsorbable component comprises a thickness of about 0.5 mm to about 5 mm.

19. The method of claim 11, wherein the plurality of one or more of nanoparticles and salts comprise one or more of a metal cation and/or alloys thereof.

20. The method of claim 11, wherein the bioabsorbable component further comprises a plurality of struts disposed within the open cell matrix.
